(19) United States
(12) Patent Application Publication
(10) Pub. No.: US 2010/0303830 A1
(43) Pub. Date: Dec. 2, 2010

(54) PREVENTION AND TREATMENT OF OTITIS MEDIA USING IgA ENRICHED MILK

(75) Inventors: Harald Bruessow, La Tour-de-Peilz (CH); Nanda De Groot, Leiden (NL); Marie-Claire Fichot, Blonay (CH)

Correspondence Address: K&L Gates LLP, P.O. Box 1135, CHICAGO, IL, 60690, US

(73) Assignee: NESTEC S.A., Vevey (CH)

(21) Appl. No.: 12/747,444

(22) PCT Filed: Dec. 8, 2008
(86) PCT No.: PCT/EP08/67027

§ 371 Date: Jul. 29, 2010

(30) Foreign Application Priority Data
Dec. 11, 2007 (EP) 07122885.2

(51) Int. Cl. A61K 39/40; A61P 27/16

(52) U.S. Cl. 424/164.1

(57) ABSTRACT

This invention relates to a composition suitable for use in the prevention or treatment of otitis media comprising IgA derived from mature bovine milk and having specificity for at least one of Streptococcus pneumoniae, Haemophilus influenzae and Moraxella catarrhalis. The invention further extends to the use of such a composition in the prevention or treatment of otitis media.

### FIELD OF THE INVENTION

This invention relates to the prevention and treatment of otitis media, particularly in infants and small children.

### BACKGROUND OF THE INVENTION

Infections of the respiratory tract are very common, particularly in infants and small children. For example, in the first year of life, an infant will often experience from three to six such infections. Such infections may be of bacterial or viral origin. Examples of viral infections of the respiratory tract include the common cold, influenza and respiratory syncytial virus. Examples of bacterial infections of the respiratory tract include pneumonia and otitis media.

Frequent respiratory tract infections are often associated with acute otitis media. This is an infection of the middle ear in which the Eustachian tube which connects the cavity of the middle ear with the external environment via the mouth becomes inflamed and then blocked trapping bacteria in the middle ear. The middle ear cavity also becomes inflamed with a build up of fluid leading to increased pressure which is experienced by the patient as pain due to the inability to equalise pressure between the middle ear and the external environment via the Eustachian tube as in healthy subjects. In severe cases, the tympanic membrane may burst under pressure allowing the infected liquid to drain to the exterior. This is a potentially dangerous situation which can lead to permanently impaired hearing if the membrane does not heal cleanly. On the other hand, if the membrane does not burst, the result may be mastoiditis, a serious complication of otitis media in which the mastoid process, a portion of the temporal bone of the skull becomes infected or inflamed. If left untreated, this can lead to meningitis or the formation of a brain abscess.

50% of children will have had at least one episode of acute otitis media in the first year of life and 35% of children between one and three years of age have recurrent episodes of acute otitis media. This in turn may lead to the development of a condition called glue ear in which the fluid does not completely drain from the middle ear between bouts of infection. If this condition becomes established, surgical intervention may be necessary.

Acute otitis media is linked to the activity of pathogenic bacteria commonly found in the indigenous microbiota of the naso-pharyngeal cavity. Quantitatively, the most important pathogens are Streptococcus pneumoniae (35% of cases), non-typeable Haemophilus influenzae (30% of cases) and Moraxella catarrhalis (10% of cases). For this reason, acute otitis media is commonly treated by the administration of antibiotics especially in infants. Indeed, antibiotics are prescribed more frequently for treatment of otitis media than for any other illness in infancy. This has inevitably led to the development of resistance to the commonly prescribed antibiotics in the bacterial strains associated with otitis media. For example, it is thought that at least 20% of S. pneumoniae strains are resistant to penicillins and cephalosporins. Similarly, at least 30% of H. influenzae strains and the majority of M. catarrhalis strains have developed antibiotic resistance. This frequency of prescription is at least in part due to the pain experienced by infants and young children suffering from otitis media to which they react by prolonged crying which parents and other care givers are very anxious to relieve. There is thus clearly a need for alternative methods to decrease the incidence of this painful and potentially serious condition in infants and young children.

It has been known for some time that human milk has a protective effect against otitis media and it is thought that this is due to specific immunoglobulins in the milk. It has been suggested, for example by Harabuchi et al, that the protective effects of human milk against otitis media may be due in part to inhibition of nasopharyngeal colonisation with nontypeable H. influenzae by secretory IgA antibodies in the milk (J Pediatr. 1994 February; 124(2): 193-8).

Various therapies based on this hypothesis have already been proposed. For example, in WO 97/17089 it is proposed to use a so-called immune milk preparation for the prevention of otitis media. This preparation contains immunoglobulins of the IgG type directed against otitis media pathogens and obtained from bovine colostrum to complement the passive immune defense.

More recently, in WO2006/022543 it is proposed to use a combination of a galactose-containing oligosaccharide and an immunoglobulin having activity against pathogenic microorganisms in the prevention or treatment of viral infections such as are caused by respiratory syncytial virus and rotavirus. According to the inventors of WO2006/022543, RSV may cause Eustachian tube dysfunction resulting in transient negative pressure in the middle ear thus facilitating secondary bacterial infections of the ear such as otitis media. WO2006/022543 also proposes the use of immunoglobulins derived from bovine colostrum or milk, preferably a mixture of IgG and IgA produced by cows immunised with a respiratory virus antigen.

From the foregoing, it may be seen that there still remains a need for an effective method for the prevention and treatment of acute otitis media which does not rely on the use of antibiotics and which may be conveniently, safely and economically administered.

### SUMMARY OF THE INVENTION

Accordingly, the present invention provides a composition suitable for use in the prevention or treatment of otitis media comprising IgA derived from mature bovine milk and having specificity for at least one of Streptococcus pneumoniae, Haemophilus influenzae and Moraxella catarrhalis.

The invention extends to the use of IgA derived from mature bovine milk and having specificity for at least one of Streptococcus pneumoniae, Haemophilus influenzae and Moraxella catarrhalis in the manufacture of a medicament or therapeutic nutritional composition for the prevention or treatment of otitis media.

The invention further extends to a method for the prevention or treatment of otitis media comprising administering to an individual in need thereof a therapeutic amount of IgA derived from mature bovine milk and having specificity for at least one of Streptococcus pneumoniae, Haemophilus influenzae and Moraxella catarrhalis.

The invention also includes mature bovine milk having a concentration of IgA specific for at least one of Streptococcus pneumoniae, Haemophilus influenzae and Moraxella catarrhalis of at least 1.5 μg/ml, preferably at least 2.5 μg/ml as well as a whey fraction derived from such mature bovine milk. The mature bovine milk (or whey fraction as the case may be) is obtainable from a cow that has been hyper-immunised for the said pathogen(s) wherein hyperimmunising comprises administering the pathogen(s) via an intramucosal route selected from an airway of the cow, intra-vaginal, intra-rectal and intra-nasal as well as to a mammary gland and/or a supra-mammary lymph node of the cow. The invention further extends to the use of such mature bovine milk and/or such whey fraction in the manufacture of a medicament or therapeutic nutritional composition for the prevention or treatment of otitis media.

Preferably, the composition comprises IgA having specificity for at least two of Streptococcus pneumoniae, Haemophilus influenzae and Moraxella catarrhalis, more preferably for all three. Most preferably, the composition comprises IgA having specificity for all three of Streptococcus pneumoniae, Haemophilus influenzae and Moraxella catarrhalis in an amount of at least 10 μg/ml.

Without wishing to be bound by theory, the inventors believe that the effectiveness of the milk-derived antibodies as described above in the prevention and treatment of otitis media may be due to the fact that antibodies of the IgA class are more appropriate to target the pathology of the disease. It is the IgAs in human milk that have been potentially associated with its protective effect against otitis media, a theory that accords with the fact that it is the IgA class of immunoglobulins that is known to be associated with the protection of mucous membranes such as those in the naso-pharyngeal region against colonisation by pathogenic bacteria such as S. pneumoniae, H. influenzae and M. catarrhalis.

It is already known, for example from UK Patent No. 1573995, that immunoglobulins may be obtained from the colostrum and milk of hyperimmunised cows. However, secretion of colostrum lasts only for 2 to 3 days after each calving and, as soon as the cows enter into a mature lactation period, levels of immunoglobulins drop sharply. Further, the immunoglobulins in colostrum are predominantly of the IgG class. Thus, colostrum is not an economically viable source of antibodies of the IgA class. Further, from a practical point of view, bovine colostrum is not a suitable source of immunoglobulins of any class for administration to human infants, for example in an infant formula or similar nutritional composition. For example, in Germany the sale of bovine colostrum for the manufacture of food products is prohibited by law. This is because bovine colostrum is not widely approved as a starting material for preparing human foodstuffs as it contains many hormones and may also contain antibiotics. Suppliers of colostrum do not know the hormone or antibiotic content of their products.

### BRIEF DESCRIPTION OF THE DRAWINGS

FIG. 1 shows the specific SIgA titre targeting whole bacterial cells as measured by whole-cell ELISA;

FIG. 2 shows the specific SIgA antibody response to the mix S. pneumoniae cells and the mix CPS of the four S. pneumoniae serotypes;

FIG. 3 shows anti-S. pneumoniae cell serotype specific SIgA levels in pooled mature milk;

FIG. 4 shows anti-S. pneumoniae CPS specific SIgA levels in pooled mature milk;

FIG. 5 shows % reduction of acute otitis media detected in a mouse model of otitis media over the period from 0 to 80 hours after challenge with S. pneumoniae.

### DETAILED DESCRIPTION OF THE INVENTION

In this specification, the following expressions have the following meanings:—

“infant” means a child under the age of 12 months;
“infant formula” means a foodstuff intended for particular nutritional use by infants during the first four to six months of life and satisfying by itself the nutritional requirements of this category of persons.
“follow-on formula” means a foodstuff intended for particular nutritional use by infants aged over four months and constituting the principal liquid element in a progressively diversified diet of this category of persons.
“growing up milk” means a milk based beverage adapted for the specific nutritional needs of young children;
“IgA specific for pathogens associated with the development of otitis media” includes IgA specific for the pathogens themselves as well as for toxins produced by the pathogens such as pneumolysin in the case of Streptococcus pneumoniae;
“pathogens associated with the development of otitis media” means one or more of Streptococcus pneumoniae, non-typeable Haemophilus influenzae and Moraxella catarrhalis;
“prevention of otitis media” means prevention of establishment of otitis media and includes reduction of risk of such establishment;
“young child” means a child between the age of one and six years.

All percentages are by weight unless otherwise stated.

As discussed above, the present invention concerns IgA derived from mature bovine milk and exhibiting specificity for the pathogens associated with the development of otitis media. Such IgA may be obtained for example from the mature milk of cows hyperimmunised according to the process described by van Dissel et al. (J. Med. Microbiol. 54, 197-205) the contents of which are incorporated herein in their entirety by reference. Briefly, the cows are mucosally immunized with a specifically designed immune stimulant. The immunization method is based on mucosal stimulation of the immune system and includes a priming immunisation which is given intra-nasally and a boost immunization that is given locally via the supra-mammary lymph node. The immune stimulation is maintained by nasal (mucosal, every two weeks), subcutaneous (every two months) and supra-mammary lymph node administration (percutaneous; once every month) which are carried out during the lactation period when the cow gives mature milk beginning 4-6 weeks after calving. This method of immunization increases the IgA level specifically in the milk of the cows throughout the lactation period.

Whey prepared from the milk of cows thus immunized contains high concentrations of specific antibodies against pneumolysin as well as against whole bacterial cells. The immunization protocol predominantly enhanced the specific IgA response in the mature milk. Indeed, the specific IgA concentration of the immune milk reached at least the concentration in pooled colostrums of identically immunized cows (that of IgG about one-tenth of that concentration). In immunized cows, high titers were maintained throughout the period of mature milk production. This enabled the collection of large amounts of immune milk and therefore the readily available mature milk rather than the briefly appearing colostrum could be used as starting material for production of immune whey and food compositions containing it.

A method of obtaining a whey protein fraction from the milk of the immunized cows using standard techniques currently employed in the dairy industry is also described in van Dissel et al. Briefly, milk from immunised cows is stored at <8° C. and pasteurised within 24 hours of receipt. The temperature and time for pasteurization may be selected within the ranges 63 to 72° C. and 15 seconds to 30 minutes but generally the combination of a relatively low pasteurization temperature e.g. 65° C. and a relatively long duration of treatment e.g. 10 minutes is preferred as this causes less deterioration of the IgA. Within 48 hours of pasteurization, the fat is removed by centrifugation and casein is removed either by acidification or enzymatically using rennet. The resulting whey fraction is pasteurized, concentrated by ultrafiltration to achieve the desired protein concentration and spray dried. Alternatively, the casein may be removed by micro-filtration techniques as is also known in the art. The use of microfiltration has the advantage that the resulting whey fraction is almost sterile. Again, the protein content may be concentrated as desired.

Preferably, the composition is a nutritional composition which is consumed as a liquid and is suitable for consumption by infants and young children. The composition may be a nutritionally complete formula such as an infant formula, a follow-on formula or a growing up milk. Alternatively for the older end of the target group of infants and young children, the composition may be a juice drink or other chilled or shelf stable beverage or a soup, for example.

Preferably a nutritional composition according to the invention contains from 3 to 150 μg/g (dry weight) of IgA specific for pathogens associated with the development of otitis media.

The general composition of an infant formula according to the invention will now be described by way of example. The formula contains a protein source. Typically, the protein source in infant formula is based on whey or a mixture of whey and casein. In infant formulas of this type, the IgA may be incorporated in the infant formula in the desired amount by replacing all or part of the whey content by a whey fraction produced from mature bovine milk according to the invention as described above. Alternatively, if skimmed milk is used as a source of whey and casein proteins in the infant formula, part or all of this may be replaced by immune milk produced as described above without the need to further process the immune milk to prepare a whey fraction. A further alternative is to replace some or all of the skimmed milk with a milk protein concentrate prepared from immune milk. Otherwise, the type of protein is not believed to be critical to the present invention provided that the minimum requirements for essential amino acid content are met and satisfactory growth is ensured. Thus, if it is preferred not to include whey protein in the infant formula or if the formula is based on hydrolysed whey protein, the IgA may be isolated from mature bovine milk according to the invention for example by sequential micro- and ultra-filtration steps or by ion exchange chromatography and added as such. In this case, protein sources based on casein and soy may be used as may partially hydrolysed whey proteins.

An infant formula according to the present invention contains a carbohydrate source. Any carbohydrate source conventionally found in infant formulae such as lactose, saccharose, maltodextrin, starch and mixtures thereof may be used although the preferred source of carbohydrates is lactose. Preferably the carbohydrate sources contribute between 35 and 65% of the total energy of the formula.

An infant formula according to the present invention contains a source of lipids. The lipid source may be any lipid or fat which is suitable for use in infant formulas. Preferred fat sources include palm olein, high oleic sunflower oil and high oleic safflower oil. The essential fatty acids linoleic and α-linolenic acid may also be added as may small amounts of oils containing high quantities of preformed arachidonic acid and docosahexaenoic acid such as fish oils or microbial oils. In total, the fat content is preferably such as to contribute between 30 to 55% of the total energy of the formula. The fat source preferably has a ratio of n−6 to n−3 fatty acids of about 5:1 to about 15:1; for example about 8:1 to about 10:1.

The infant formula will also contain all vitamins and minerals understood to be essential in the daily diet and in nutritionally significant amounts. Minimum requirements have been established for certain vitamins and minerals. Examples of minerals, vitamins and other nutrients optionally present in the infant formula include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form. The presence and amounts of specific minerals and other vitamins will vary depending on the intended infant population.

If necessary, the infant formula may contain emulsifiers and stabilisers such as soy lecithin, citric acid esters of mono- and di-glycerides, and the like.

The infant formula may optionally contain other substances which may have a beneficial effect such as probiotics, lactoferrin, nucleotides, nucleosides, and the like.

Finally, the formula will contain from 3 to 150 μg IgA derived from mature bovine milk and exhibiting specificity for the pathogens associated with the development of otitis media per gram of infant formula powder.

The formula may be prepared in any suitable manner having regard to the fact that IgA may be denatured by temperatures in excess of 60° C. For example, the formula may be prepared by blending together the carbohydrate source and the fat source in appropriate proportions. If used, the emulsifiers may be included at this point. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently about 50° C. to about 80° C. to aid dispersal of the ingredients. Commercially available liquefiers may be used to form the liquid mixture. The liquid mixture is then homogenised; for example in two stages.

The liquid mixture may then be thermally treated to reduce bacterial loads, by rapidly heating the liquid mixture to a temperature in the range of about 80° C. to about 150° C. for about 5 seconds to about 5 minutes, for example. This may be carried out by steam injection, autoclave or by heat exchanger; for example a plate heat exchanger.

Then, the liquid mixture may be cooled to about 60° C. to about 85° C.; for example by flash cooling. The liquid mixture may then be again homogenised; for example in two stages at about 10 MPa to about 30 MPa in the first stage and about 2 MPa to about 10 MPa in the second stage. The homogenised mixture may then be further cooled to add any heat sensitive components; such as vitamins and minerals. The pH and solids content of the homogenised mixture are conveniently adjusted at this point.

The homogenised mixture is transferred to a suitable drying apparatus such as a spray drier or freeze drier and converted to powder. The powder should have a moisture content of less than about 5% by weight. This powder is then dry-mixed with a whey fraction obtained from mature bovine milk according to the invention by the processes of ultra-filtration and spray drying as is known to those skilled in the art. The whey fraction should meet the microbiological criteria for addition by dry mixing.

In another embodiment, the composition may be a supplement including the IgA in an amount sufficient to achieve the desired effect in an individual. This form of administration is more suited to children at the upper end of the target age group. Preferably the daily dose of the IgA is between 2500 and 30000 μg. The amount of IgA to be included in the supplement will be selected accordingly depending upon how the supplement is to be administered. For example, if the supplement is to be administered twice a day, each supplement will contain from 1250 to 15000 μg of antibodies. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or enteral administration as well as vitamins, minerals trace elements and other micronutrients in accordance with the recommendations of Government bodies such as the USRDA.

### Example 1

An example of the composition of an infant formula according to the present invention is given below. This composition is given by way of illustration only.

| Nutrient | per 100 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Prebiotic (70% FOS, 30% | 0.64 | 4.3 |
| inulin) (g) | | |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |

| | |
|---|---|
| Specific IgA | 10 μg/ml of ready to consume formula |

### Example 2

### Preparation of Immune Stimulant

The following pathogen strains were selected to prepare the immune stimulant:—Streptococcus pneumoniae serotypes 23F (ATCC 700669), 19F (ATCC 700905), 6B (ATCC 700670) and 9V (ATCC 700671) and non-virulent non-encapsulated R6 strain, Haemophilus influenzae (040921 clinical isolate) and Moraxella catarrhalis (035E wild type isolate middle ear). The strains were cultured on a more defined medium lacking serum and animal tissue derived components. The medium basic components are yeast extract and soya peptone—papaic digest buffered with phosphate and bicarbonate (pH 7.4). 1% (w/v) glucose was added to the medium for culture of S. pneumoniae and 1% (w/v) glucose, 15 mg/l Hemin and 15 mg/l NAD were added to the medium for culture of H. influenzae. The strains were cultured for 10 to 15 hours.

The bacterial cell component was inactivated by treating with 0.37% (v/v) formaldehyde at 37° C. typically for 6 days or heat typically at 70° C. for 2 hours. Formaldehyde was removed by diafiltration to a final concentration below 0.2% (w/v).

The supernatant of the bacterial cultures or cell lysates were used as a source of secreted bacterial protein products. It was inactivated by treating with 0.37% (v/v) formaldehyde at 37° C. typically for 6 days. Formaldehyde was removed by diafiltration (final concentration below 0.2% (w/v)).

The cell component and the protein component were combined to produce an immune stimulant containing whole cell pathogens associated with the development of otitis media and their adherence/virulence antigens.

### Example 3

### SIgA Specific Antibody Titre Targeting Streptococcus pneumoniae, Haemophilus influenzae and Moraxella catarrhalis

Healthy dairy cows were mucosally immunized according to Van Dissel et al. using the immune-stimulant described in Example 2 above.

Specific SIgA antibody titres were measured by ELISA. The ELISA standard was prepared from the first 3-51 of colostrum from a separate group of 4 cows immunized according to Van Dissel et al. using the immune-stimulant described in Example 2 above during their late pregnancy and is expressed in units/ml, based on the assumption that undiluted standard preparation is 1000 units/ml. Whey sample specific antibody values are expressed in units/ml and compared to the levels within the standard preparation.

Briefly, the ELISA was performed as follows. Assays were optimized using the standard preparation and per assay one combination of three variables e.g. coating antigen dilution, Moab-anti-bovine IgA-dig dilution and HRP-labelled sheep anti-dig (Roche) dilution was selected as optimized setting. For coating, the antigens were 1) whole S. pneumoniae cells, optimized dilution per serotype and mix (1:1) serotypes; 2) whole M. catarrhalis cells (035E clinical isolate middle ear); 3) whole H. influenzae cells (040921 clinical isolate); 4) Capsular Polysaccharides—CPS, single CPS 19F/23F/6B/9V and 9N/14 at a concentration of 10 μg/ml or mixed 1:1 CPS (19F/23F/6B/9V total concentration 10 μg/ml).

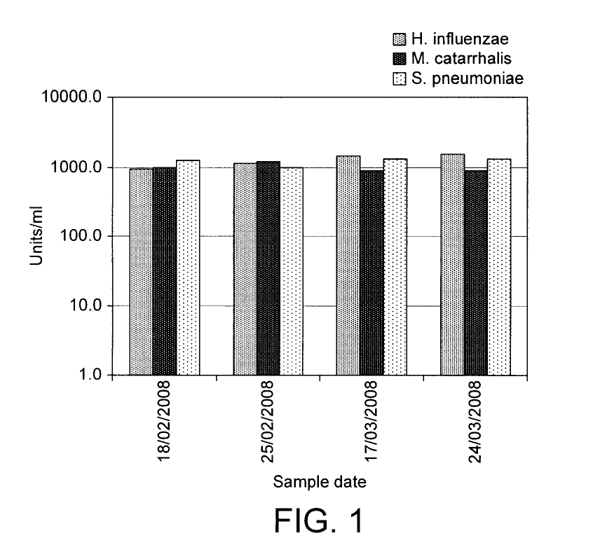

FIG. 1 shows the specific SIgA titre targeting whole bacterial cells as measured by whole-cell ELISA. The titres were measured in whey of mature milk obtained from 12 individual immunized cows during a two months time-period. It may be seen that average specific anti-S. pneumoniae SIgA antibody titres reached the level found in colostrum from identically immunized cows. The level of specific SIgA antibody is maintained during the lactation period (FIG. 1 shows two consecutive months). Similar data was obtained for specific antibody levels targeting H. influenzae and M. catarrhalis whole cells.

Background antibody levels in non-immune whey are low, and mostly below the detection level of the ELISA assay.

SIgA Specific Antibody Titre Targeting Streptococcus pneumoniae, Serotype Specific

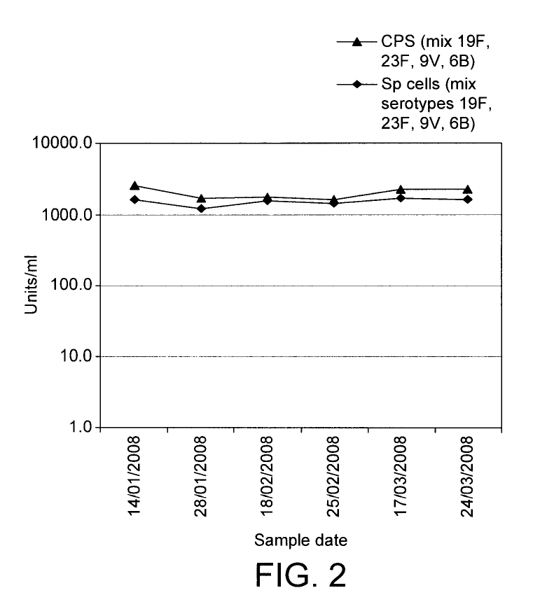

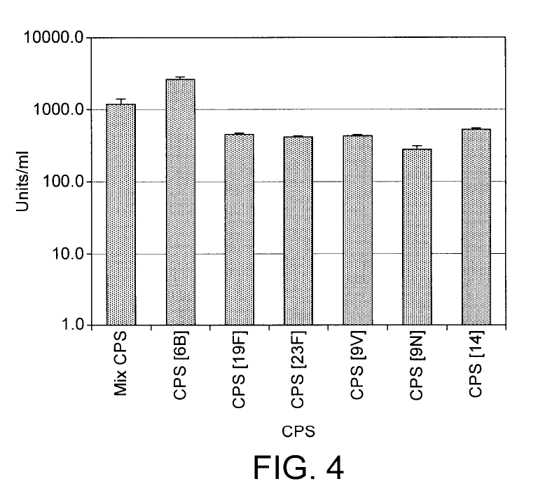

The antibody response towards the specific capsular polysaccharides (CPS) was also measured directly using purified polysaccharides (19F, 23F, 6B, 9V, 9N and 14 obtained from ATCC) as coating antigens. The four CPS within the immune-stimulant were used as coating antigens mixed (1:1 concentration 10 μg/ml) (FIG. 2) or as single CPS (10 μg/ml) (FIG. 4). Also two CPS (9N and 14), not present within the immune-stimulant, were tested within ELISA to show the level of cross-reactivity.

FIG. 2 shows the specific SIgA antibody response to the mix S. pneumoniae cells and the mix CPS of the four S. pneumoniae serotypes. The data represents the average specific antibody level in whey of mature milk obtained from a group of 12 individual immunized cows during their lactation period for the first 3 months of 2008.

Background antibody levels in non-immune whey are low, around 0.5-1.0 units/ml.

Specific anti-S. pneumoniae serotype specific SIgA was raised during immunization throughout the lactation period of the cows. As may be seen from FIG. 2, high specific antibody responses were maintained in the milk of the 12 immunized cows for two consecutive months.

Anti-S. pneumoniae Cell Serotype Specific and CPS Specific SIgA Levels in Pooled Mature Milk

An antibody preparation was prepared from a pool of milk obtained from 12 immunized cows during the month of April 2008. The pooled immune milk from 12 cows was collected on one day and a whey preparation, including concentrated fractions, was prepared. The specific SIgA antibody whey fraction was analysed on antibody specificity and bioactivity in vitro (agglutination assay) and in vivo (murine model for otitis media).

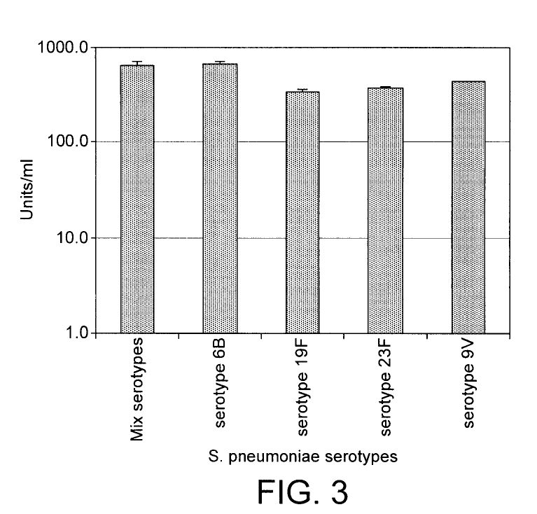

FIGS. 3 and 4 give an overview of the type specific antibody response towards S. pneumoniae serotypes and CPS of the pooled immune whey fraction (performed in quadruplicate and duplicate respectively). To measure the antibody response towards the individual specific serotypes more precisely, whey samples were pre-incubated (60 minutes, 37° C.) with purified commercial C-polysaccharide (Statens Serum Institute) and afterwards tested in the CPS specific ELISA. Depletion of the antibody fraction for the C-polysaccharide was checked in ELISA with C-polysaccharide as coating antigen.

SIgA anti-S. pneumoniae whole cells (mix) and CPS (mix) levels in the whey preparation are around 1000 units/ml (as indicated also in FIG. 2). SIgA specific antibody response towards serotypes 23F, 19F and 9V are equally high, indicating that no real difference in antigenicity exists between these three serotypes. Response towards serotype 6B is the highest of all four implying that this is the most dominant antigen (FIGS. 3 and 4). Cross-reactivity with two other CPS types (9N, 14) not included within the immune-stimulant is present within the specific antibody whey preparation (FIG. 4), indicating that the immune-stimulant raises a broad polyclonal antibody response in the immunized cows recognizing more different type specific CPS.

### Functional Activity of the Specific Anti-S. Pneumoniae SIgA Antibodies In Vitro (Agglutination Assay) and In Vivo (Murine Model of Otitis Media)

Interaction between specific cell wall targeted antibodies and whole bacterial cells is one of the important features in preventing bacteria from colonizing the mucosal surface by binding to epithelial cells. In the agglutination assay the induction of agglutination of S. pneumoniae bacterial cells was measured by testing the specific targeted whey-derived antibody preparations. The assay was performed in a 96 well set-up using S. pneumoniae serotype 19F. Briefly, S. pneumoniae serotype 19F (formaldehyde inactivated) at a fixed bacterial density of 1.0 (OD600 nm) was used. PBS, control milk whey and milk whey test sample preparations were two-fold serial diluted within the 96-well set-up with a final volume of 50 μl per well. 50 μl of bacterial culture was added to each well of the plate and incubation was carried out overnight at 4° C. Plates were scored indicating the highest sample dilution that showed agglutination of the bacterial cells.

Table 1 below shows the results from the agglutination assay. Non-immune whey shows a low background signal, which is not detectable at >2-fold sample dilution. The immune whey sample prepared from the pooled milk has on average an agglutination titre of 8-fold dilution. For the milk from the individual cows the agglutination titre varied between 8-fold and 64-fold dilution, indicating the difference between milk of individual cows and the importance of the polyclonal feature of the antibody preparation.

TABLE 1

| | Agglutination | Total protein content |
|---|---|---|
| | titre | (mg/ml) |
| Immune whey (pool milk, | 16 | 10.8 |
| 12 cows) | | |
| Agglutination titre | 8 to 64 | |
| (individual cows, 12) | | |
| Non-immune whey (pool | 2 | 9.3 |
| milk, 3 cows) | | |

For the in vivo investigation, the murine model of otitis media developed by McCullers et al was used (McCullers et al, “Novel Strategy to Prevent Otitis Media Caused by Colonising Streptococcus pneumoniae” PLoS Pathogens, March 2007, Vol 3, Issue 3). Briefly, groups of mice maintained in a BL2 facility were infected intra-nasally with 10e5 or 10e6 CFU of a piliated strain of S. pneumoniae known to efficiently colonise mucosal surfaces (a type 19F strain obtained from B. Henriques-Normark ST162 19F) engineered to express luciferase. Animals were followed daily for development of infection for two weeks and thrice weekly for another four weeks. Within 72 hours of pneumococcal infection, all the mice were visibly colonised with bacteria in the anterior portion of the nose and 70% had developed acute otitis media (AOM). These infections of the middle ear all resolved by bioluminescent imaging within 48 hours and no mice had evidence of AOM six days after challenge or later. Nasal colonisation persisted for a median of 27 days.

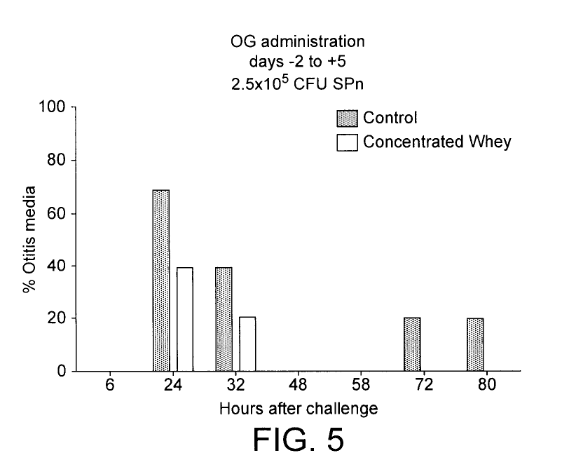

This model was used to evaluate the efficacy of the same whey-derived antibody preparation according to the invention as was tested in the in vitro assay. Two days prior to challenge with 2.5×10e5 CFU of the bioluminescent S. pneumoniae, the mice received 100 μl of antibody preparation or control whey preparation by oral gavage (n=10 in each group). Treatment continued daily for seven days. The results are shown in FIG. 5 from which it may be seen that there was a reduction in otitis media in the experimental group.

### Example 4

### Preparation of Whey Fraction

Mature milk was obtained from cows immunised with the immune stimulant described in Example 2. The milk was stored at a temperature below 8° C. and was pasteurised (10 minutes at 65° C.) within 24 hours of receipt. A whey fraction enriched in IgA specific for the strains used in preparation of the immune stimulant was obtained by centrifuging the milk to remove fat and removing the casein by microfiltration. The whey fraction thus obtained was pasteurised again using the same conditions, concentrated by ultrafiltration and spray dried to produce a powder. The whey fraction had a protein content of about 40% of which about 10% was immunoglobulins.

1. A composition suitable for use in the prevention or treatment of otitis media comprising IgA derived from mature bovine milk and having specificity for at least one pathogen selected from the group consisting of Streptococcus pneumoniae, Haemophilus influenzae and Moraxella catarrhalis.

2. A composition according to claim 1 wherein the IgA is specific for all of Streptococcus pneumoniae, Haemophilus influenzae and Moraxella catarrhalis.

3. A composition according to claim 1, which is a nutritional composition.

4. A composition according to claim 1 which is in a form selected from the group consisting of an infant formula, a follow-on formula and a growing-up milk.

5. A composition according to claim 4 comprising from 3 to 150 μg of IgA per gram of composition on a dry weight basis.

6. A composition according to claim 1 which is a supplement and which comprises from 2500 to 30000 μg of IgA per daily dose.

7. A method for the prevention or treatment of otitis media comprising administering a composition comprising IgA derived from mature bovine milk and having specificity for at least one pathogen selected from the group consisting of Streptococcus pneumoniae, Haemophilus influenzae and Moraxella catarrhalis to an individual in need of same.

8. Mature bovine milk having a concentration of IgA of at least 1.5 μg/ml specific for at least one pathogen selected from the group consisting of Streptococcus pneumoniae, Haemophilus influenzae and Moraxella catarrhalis.

9. Mature bovine milk according to claim 8 having a concentration of the IgA of at least 2.5 μg/ml.

10. Mature bovine milk according to claim 8 which has a concentration of IgA of at least 10 μg/ml specific for all three of Streptococcus pneumoniae, Haemophilus influenzae and Moraxella catarrhalis.

11. Mature bovine milk according to claim 10 which has a concentration of IgA of at least 12 μg/ml specific for all three of Streptococcus pneumoniae, Haemophilus influenzae and Moraxella catarrhalis.

12. A whey fraction produced from mature bovine milk having a concentration of IgA of at least 1.5 μg/ml specific for at least one pathogen selected from the group consisting of Streptococcus pneumoniae, Haemophilus influenzae and Moraxella catarrhalis.

13. A method according to claim 7 wherein the IgA is specific for all of Streptococcus pneumoniae, Haemophilus influenzae and Moraxella catarrhalis.

14. A method according to claim 7, which is a nutritional composition.

15. A method according to claim 7 which comprises from 3 to 150 μg of IgA per gram of composition on a dry weight basis.

16. A method according to claim 7 which, is a supplement and which comprises from 2500 to 30000 μg of IgA per daily dose.

17. A method according to claim 7 wherein the composition is in a form selected from an infant formula, a follow-on formula and a growing-up milk.
